(19) United States
(12) Patent Application Publication
(10) Pub. No.: US 2024/0074696 A1
(43) Pub. Date: Mar. 7, 2024

(54) INFORMATION PROCESSING DEVICE, INFORMATION PROCESSING METHOD, AND STORAGE MEDIUM

(71) Applicant: CANON MEDICAL SYSTEMS CORPORATION, Otawara-shi (JP)

(72) Inventors: Kosuke ARITA, Nasushiobara-shi (JP); Yudai YAMAZAKI, Nasushiobara-shi (JP)

(73) Assignee: CANON MEDICAL SYSTEMS CORPORATION, Otawara-shi (JP)

(21) Appl. No.: 18/459,550

(22) Filed: Sep. 1, 2023

(30) Foreign Application Priority Data
Sep. 5, 2022 (JP) 2022-140668

(51) Int. Cl. A61B 5/00; G06V 10/44

(52) U.S. Cl. CPC A61B 5/441; A61B 5/0033; A61B 5/0075; G06V 10/44

(57) ABSTRACT

An information processing device includes processing circuitry. The processing circuitry acquires observation data which is acquired when a target event is observed. The processing circuitry converts the observation data to a feature quantity of the target event using a machine learning model. The processing circuitry restores the observation data from the feature quantity using a numerical simulation model. The processing circuitry trains the machine learning model on the basis of a discrepancy between first observation data which is the observation data that has not been converted to the feature quantity and second observation data which is the observation data restored from the feature quantity.

### CROSS-REFERENCE TO RELATED APPLICATION

The present application claims priority based on Japanese Patent Application No. 2022-140668, filed Sep. 5, 2022, the content of which is incorporated herein by reference.

### FIELD

Embodiments disclosed in this specification and the accompanying drawings relate to an information processing device, an information processing method, and a storage medium.

### BACKGROUND

An edema is an important observation for various diseases such as cardiac insufficiency or deep vein thrombosis. The primary disease of an edema needs to be identified to carry out appropriate treatment. In general, a doctor makes a diagnosis on the basis of information acquired through visual inspection or palpation. However, diagnosis accuracy depends greatly on a doctor's subjective determination based on their medical knowledge or skill. In order to solve this problem, a method of analyzing an image acquired from a camera can be used, and information associated with an edema can be noninvasively and simply quantified using this method.

A method in which simulation and machine learning are combined is known as an edema recognition method using an optical camera. For example, it is conceivable to estimate an input parameter (an in-vivo component) of simulation from an image using a machine learning model called an autoencoder. However, there may be a discrepancy between simulation and an actual phenomenon, but an in-vivo component cannot be correctly estimated when the autoencoder is trained to restore an observation value.

As a method of training an autoencoder, for example, a method of training an autoencoder on the basis of a loss function including errors of image feature quantities or a method of generating a latent variable by dimensionally compressing input data using a variational autoencoder in which an encoder and a decoder are combined, generating output data by restoring the latent variable, generating a latent variable by dimensionally compressing output data using another encoder, and training the variational autoencoder on the basis of a loss function including an error between two latent variables is known.

However, when these methods are applied to a situation in which there is a discrepancy between an actual phenomenon (an observation value) and a simulation result (a restored value), training may be performed while ignoring the discrepancy. As a result, a condition different from an actual patient condition may be estimated, which may lead to erroneous recognition of an edema condition.

The situation in which there is a discrepancy between an actual phenomenon and a simulation result is present in symptoms other than edemas. The situation in which there is a discrepancy between an actual phenomenon and a simulation result is not limited to the medical field, but may appear in all the fields such as physics, chemistry, engineering, biology, earth science, information, finance, and economy. Accordingly, the aforementioned problem is common to all the fields in which there may be a discrepancy between an actual phenomenon and a simulation result.

### BRIEF DESCRIPTION OF THE DRAWINGS

FIG. 1 is a diagram illustrating an example of a configuration of an information processing device according to an embodiment.

FIG. 2 is a flowchart illustrating a flow of a series of processes which are performed by processing circuitry according to the embodiment.

FIG. 3 is a diagram schematically illustrating a flow of a series of processes which are performed by the processing circuitry according to the embodiment.

FIG. 4 is a diagram illustrating a frequency distribution p(μ_{R\hat{ }-R}) of an average μ of discrepancies (R\hat{ }−R) for each pixel.

FIG. 5 is a diagram illustrating a frequency distribution p(R\hat{ }(λ)−R(λ)) of discrepancies (R\hat{ }(λ)−R(λ)) for each wavelength λ.

FIG. 6 is a flowchart illustrating a flow of a series of processes which are performed by the processing circuitry according to the embodiment.

FIG. 7 is a diagram illustrating a screen example of a display on which an in-vivo component volume map is displayed.

FIG. 8 is a diagram illustrating a screen example of a display on which an in-vivo component volume map is displayed.

FIG. 9 is a diagram illustrating a screen example of a display on which an in-vivo component volume map is displayed.

### DETAILED DESCRIPTION

Hereinafter, an information processing device, an information processing method, and a storage medium according to an embodiment will be described with reference to the accompanying drawings. The information processing device according to the embodiment acquires observation data which is acquired when a target event is observed. For example, a target event is an “edema” in the medical field, and the observation data thereof is a “spectral reflectance” which is acquired from an edema image. The target event is not limited to an edema, and may be another symptom or disease. The target event is not limited to the medical field and may be a target to be analyzed in another field such as physics, chemistry, engineering, biology, earth science, information, finance, or economy. In this embodiment, the target event is assumed to be an “edema” as an example.

When an edema image is acquired, the information processing device according to the embodiment converts a spectral reflectance (a spectral reflectance corresponding to each pixel value of the edema image) acquired from the edema image to a feature quantity of an edema using a machine learning model and restores the spectral reflectance from the feature quantity of the edema using a numerical simulation model. Then, the information processing device trains the machine learning model on the basis of a discrepancy between the spectral reflectance not having been converted to the feature quantity and the spectral reflectance having been restored from the feature quantity. By diagnosing a patient using the machine learning model having been trained in this way, it is possible to provide information with a higher certainty factor (here, a diagnosis result indicating whether an edema is present in a living body) even when there is a discrepancy between an actual phenomenon and a simulation result.

[Configuration of Information Processing Device]

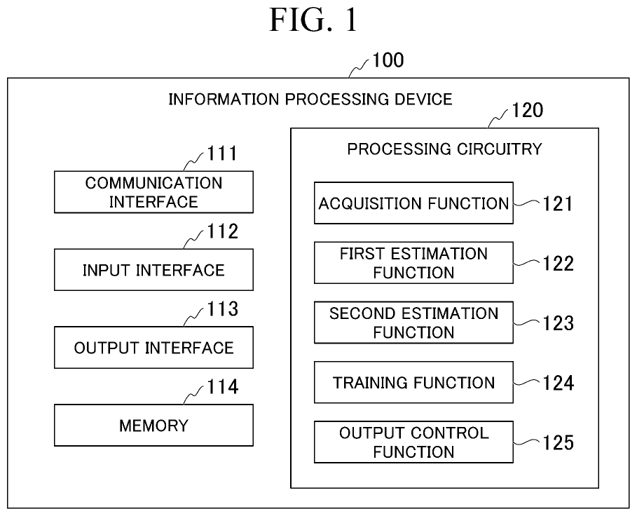

FIG. 1 is a diagram illustrating an example of a configuration of the information processing device 100 according to the embodiment. The information processing device 100 includes, for example, a communication interface 111, an input interface 112, an output interface 113, a memory 114, and processing circuitry 120.

The communication interface 111 communicates with an external device via a communication network NW. The communication network NW may be a whole information communication network using telecommunication technology. For example, the communication network NW includes a telephone communication network, an optical fiber communication network, a cable communication network, and a satellite communication network in addition to a wireless/wired local area network (LAN) such as a hospital mainstay LAN or the Internet. The communication interface 111 includes, for example, a network interface card (NIC) or an antenna for wireless communication.

The input interface 112 receives various input operations from an operator, converts the received input operations to electrical signals, and outputs the electrical signals to the processing circuitry 120. For example, the input interface 112 includes a mouse, a keyboard, a track ball, a switch, a button, a joystick, and a touch panel. The input interface 112 may be, for example, a user interface for receiving speech inputs such as a microphone. When the input interface 112 is a touch panel, the input interface 112 may have a display function of a display 113a included in the output interface 113 which will be described later.

The input interface 112 in this specification is not limited to including physical operation parts such as a mouse and a keyboard. For example, electrical signal processing circuitry for receiving an electrical signal corresponding to an input operation from an external input device which is provided separately from the device and outputting the electrical signal to a control circuit is also included in examples of the input interface 112.

The output interface 113 includes, for example, a display 113a and a speaker 113b. The display 113a displays various types of information. For example, the display 113a displays an image generated by the processing circuitry 120 or a graphical user interface (GUI) for receiving various input operations from an operator. For example, the display 113a is a liquid crystal display (LCD), a cathode ray tube (CRT) display, or an organic electroluminescence (EL) display. The speaker 113b outputs information input from the processing circuitry 120 by speech.

The memory 114 is realized, for example, by a semiconductor memory element such as a random-access memory (RAM) or a flash memory, a hard disk, or an optical disc. This non-transitory storage medium may be realized by another storage device such as a network attached storage (NAS) or an external storage server device which is connected via the communication network NW. The memory 114 may include a non-transitory storage medium such as a read only memory (ROM) or a register. Programs which are executed by a hardware processor of the processing circuitry 120, various calculation results from the processing circuitry 120, first model information, second model information, and the like are stored in the memory 114.

The first model information is information (a program or an algorithm) defining a machine learning model MDL1 which will be described later. The second model information is information (a program or an algorithm) defining a numerical simulation model MDL2 which will be described later.

The processing circuitry 120 includes, for example, an acquisition function 121, a first estimation function 122, a second estimation function 123, a training function 124, and an output control function 125. The processing circuitry 120 realizes the functions, for example, by causing a hardware processor (a computer) to execute a program stored in the memory 114 (a storage circuit). The acquisition function 121 is an example of an “acquisition unit,” the first estimation function 122 is an example of a “first conversion unit,” the second estimation function 123 is an example of a “second conversion unit,” and the training function 124 is an example of a “training unit.”

A hardware processor in the processing circuitry 120 is, for example, circuitry such as a central processing unit (CPU), a graphics processing unit (GPU), an application-specific integrated circuit (ASIC), or a programmable logic device (for example, a simple programmable logic device (SPLD), a complex programmable logic device (CPLD), or a field-programmable gate array (FPGA)). A program may be directly assembled into the circuitry of the hardware processor instead of storing a program in the memory 114. In this case, the hardware processor realizes the functions by reading and executing a program assembled into the circuitry. The program may be stored in the memory 114 in advance or may be stored in a non-transitory storage medium such as a DVD or a CD-ROM and installed in the memory 114 from the non-transitory storage medium by setting the non-transitory storage medium in a drive device (not illustrated) of the user interface 10. The hardware processor is not limited to a single circuit, and may be configured as a single hardware processor by combining a plurality of independent circuits and realize the functions. A plurality of elements may be incorporated into a single hardware processor to realize the functions.

[Process Flow and Training of Information Processing Device]

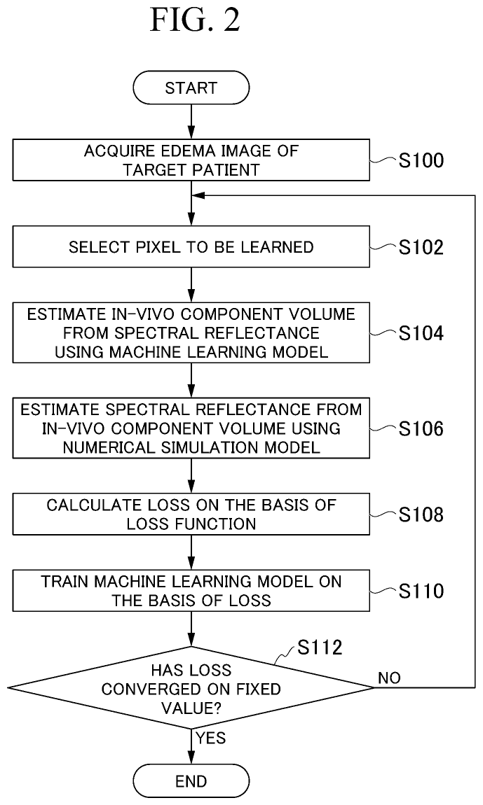

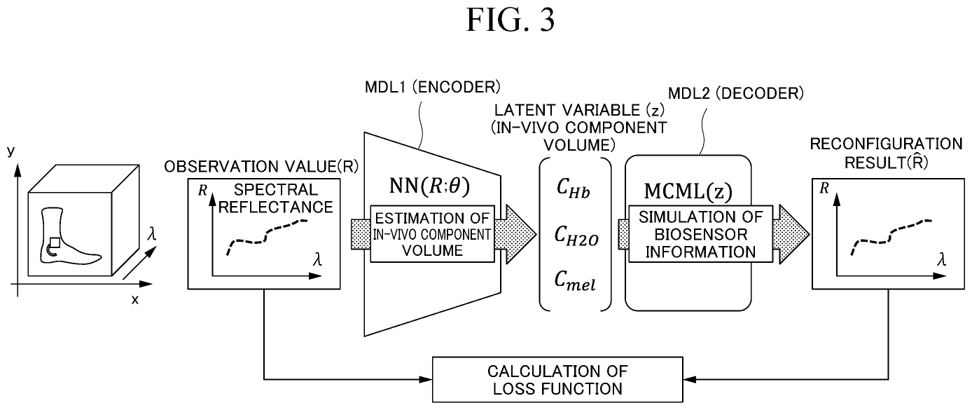

A series of processes which are performed by the processing circuitry 120 of the information processing device 100 will be described below on the basis of a flowchart. FIG. 2 is a flowchart illustrating a flow of a series of processes which are performed by the processing circuitry 120 according to the embodiment. FIG. 3 is a diagram schematically illustrating a flow of a series of processes which are performed by the processing circuitry 120 according to the embodiment. The routine of the flowchart illustrated in FIG. 2 is performed when a machine learning model MDL1 which will be described later is trained.

First, the acquisition function 121 acquires an edema image of a patient which is a training target (Step S100).

An edema image is an image obtained by imaging the skin using a camera for visualizing a spectral reflectance R of the skin in a region in which an edema is likely to be present. The edema image may be, for example, a three-dimensional image which is represented by a width x, a height y, and a wavelength λ for visualization. The pixel value of each pixel of the edema image is a spectral reflectance R. The camera used to generate an edema image is typically a multi-spectrum camera that visualizes the spectral reflectance R in a plurality of wavelength bands (spectrums), but the present invention is not limited thereto and it may be a camera that visualizes only a spectral reflectance R in a single wavelength band. An edema image with the spectral reflectance R as pixel values or the spectral reflectance R of the pixels of the edema image are an example of “observation data.”

For example, the acquisition function 121 may access a database which is an external device via the communication interface 111 and acquire an edema image from the database. When a doctor in charge of a patient or the like inputs an edema image to the input interface 112, the acquisition function 121 may acquire the edema image from the input interface 112. When an edema image is stored in the memory 114, the acquisition function 121 may acquire the edema image from the memory 114.

The acquisition function 121 may acquire an absorbance of the skin surface detected by a wearable sensor attached to a patient's arm or leg as an edema image in addition to or instead of acquisition of an edema image captured by a camera.

When an edema image is acquired, the acquisition function 121 may perform image processing such as a smoothing filter or edge extraction on the edema image. Accordingly, it is possible to remove palm prints, body hairs, or the like appearing in the edema image and to more accurately extract a feature quantity of an edema from the edema image.

Then, the first estimation function 122 selects one pixel to be learned out of a plurality of pixels included in the edema image acquired by the acquisition function 121 (Step S102).

Then, the first estimation function 122 estimates an in-vivo component volume from a pixel value, that is, a spectral reflectance R, of the pixel to be learned using a machine learning model MDL1 defined by the first model information in the memory 114 (Step S104). The in-vivo component volume is an example of a “feature quantity.”

The in-vivo component volume may include, for example, feature quantities such as C_{Hb}, CH₂O, and Cₘₑₗ. C_{Hb} is a hemoglobin concentration [g/L] included in the inner skin, CH₂O is a moisture concentration [g/L] included in the subcutaneous tissue, and Cₘₑₗ is a melanin concentration [g/L] included in the skin surface. The machine learning model MDL1 is an encoder, and an in-vivo component volume output from the encoder is a so-called latent variable z.

The first model information is information (a program or an algorithm) defining the machine learning model MDL1.

The machine learning model MDL1 may be implemented, for example, using a neural network which has been trained such that an output result of the numerical simulation model MDL2 (the spectral reflectance R\hat{ } of each pixel of the edema image of a certain patient) becomes close to the spectral reflectance R of each pixel of the edema image of the patient. The machine learning model MDL1 may be implemented using a genetic algorithm instead of the neural network. The machine learning model MDL1 may be implemented using another optimization method such as Bayes optimization, grid search, random search, CMA-ES, a Nelder-Mead method, or a quasi-Newton method. In the example illustrated in FIG. 3, the machine learning model MDL1 (encoder) is implemented using a neural network (NN(R:θ)).

When the machine learning model MDL1 is implemented using a neural network, the model information includes, for example, coupling information indicating how units included in an input layer, one or more hidden layers (intermediate layers), and an output layer of the neural network are coupled to each other or weight information indicating what a coupling coefficient applied to data input and output between the coupled units is.

The coupling information includes, for example, information indicating the number of units included in each layer or a type of a destination unit of each unit, an activation function for realizing the units, and a gate provided between the units of the hidden layers.

The activation function for realizing a unit may be, for example, a rectified linear unit (ReLU) function, an exponential linear units (ELU) function, a clipping function, a sigmoid function, a step function, a hyperbolic tangent function, or an identity function. The gate selectively passes or weights data transmitted between the units on the basis of a value (for example, 1 or 0) returned by the activation function.

The coupling coefficient includes, for example, a weight applied to output data when data is output from a unit of a certain layer to a unit of a deeper layer in the hidden layers of the neural network. The coupling coefficient may include a bias component specific to each layer.

For example, the first estimation function 122 inputs a spectral reflectance R corresponding to a pixel value of a pixel to be learned to the machine learning model MDL1. Accordingly, the machine learning model MDL1 outputs an in-vivo component volume (C_{Hb}, CH₂O, and Cₘₑₗ) in response to an input of the spectral reflectance R. That is, the first estimation function 122 converts (encodes) the spectral reflectance R corresponding to the pixel value of the pixel to be learned to the in-vivo component volume (C_{Hb}, CH₂O, and Cₘₑₗ) using the machine learning model MDL1.

Then, the second estimation function 123 estimates a spectral reflectance R\hat{ } from the in-vivo component volume (C_{Hb}, CH₂O, and Cₘₑₗ) to which the spectral reflectance R is estimated or converted by the first estimation function 122 using the numerical simulation model MDL2 defined by the second model information in the memory 114 (Step S106).

The numerical simulation model MDL2 is a model for calculating or simulating the spectral reflectance R\hat{ } of each pixel of an edema image of a certain patient from the in-vivo component volume (C_{Hb}, CH₂O, and Cₘₑₗ) of the patient. The numerical simulation model MDL2 may be implemented, for example, on the basis of a Monte Carlo method, the Kubelka-Munk theory, or the Lambert-Beer law. The numerical simulation model MDL2 is also referred to as a decoder or a simulator. In the example illustrated in FIG. 3, the numerical simulation model MDL2 (decoder) is implemented using the Monte Carlo method (MCML(z)).

For example, the second estimation function 123 inputs the in-vivo component volume (C_{Hb}, CH₂O, and Cₘₑₗ) to which the spectral reflectance R is estimated or converted by the first estimation function 122 to the numerical simulation model MDL2. Accordingly, the numerical simulation model MDL2 outputs the spectral reflectance R\hat{ } by simulation in response to an input of the in-vivo component volume (C_{Hb}, CH₂O, and Cₘₑₗ). That is, the second estimation function 123 restores (decodes) the spectral reflectance R\hat{ } from the in-vivo component volume (C_{Hb}, CH₂O, and Cₘₑₗ) using the numerical simulation model MDL2.

Then, the training function 124 calculates a loss on the basis of a certain loss function F (Step S108).

The loss function F is a function which is defined on the basis of the assumption that a frequency distribution of a discrepancy (also referred to as a difference or an error) between the spectral reflectance R before encoding using the machine learning model MDL1 has been performed and the spectral reflectance R\hat{ } after decoding using the numerical simulation model MDL2 has been performed conforms to a predetermined probability density distribution. This loss function F can be expressed, for example, by Expression (1). The spectral reflectance R before encoding using the machine learning model MDL1 has been performed is an example of “first observation data,” and the spectral reflectance R\hat{ } after decoding using the numerical simulation model MDL2 has been performed is an example of “second observation data.”

F=∥\hat{R}−R∥²+D_{KL}[p(μ_{\hat{R}-R})∥N(0,1)] (1)

The loss function F expressed by Expression (1) includes a first term in the first half stage and a second term in the second half stage. The first term represents the magnitude of the discrepancy (R\hat{ }−R) between the spectral reflectance R\hat{ } and the spectral reflectance R, and may be expressed, for example, by a mean square error or a mean absolute error. The second term represents a distance D_{KL} between distributions when an average μ_{R\hat{ }-R} of the discrepancy (R\hat{ }−R) for each pixel is calculated and it is assumed that the frequency distribution p(μ_{R\hat{ }-R}) of the average μ_{R\hat{ }-R} for all the pixels conforms to a normal distribution N(0, 1) with an average 0 and a variance 1. The distance D_{KL} between the frequency distribution p(μ_{R\hat{ }-R}) and the normal distribution N(0, 1) indicates a KL divergence distance.

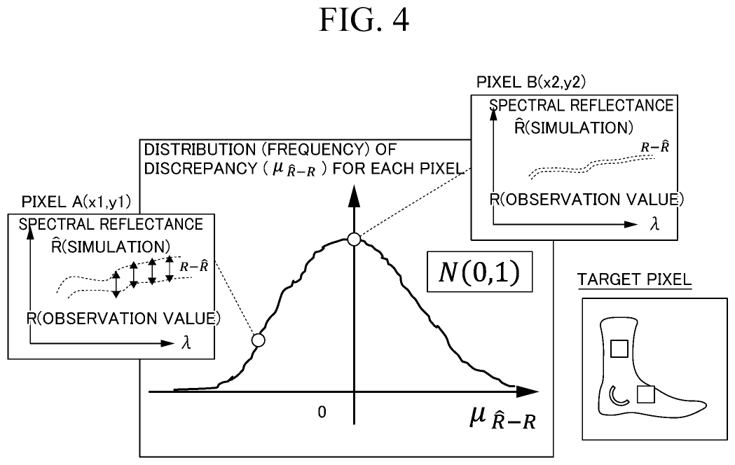

FIG. 4 is a diagram illustrating a frequency distribution p(μ_{R\hat{ }-R}) of an average μ of the discrepancy (R\hat{ }−R) for each pixel. For example, the training function 124 calculates an average μ of the discrepancy (R\hat{ }−R) based on a wavelength λ for a pixel A(x1, y1) and also calculates an average μ of the discrepancy (R\hat{ }−R) based on a wavelength λ for a pixel B(x1, y1). In the example illustrated in the drawing, it can be seen that the average μ of the discrepancy (R\hat{ }−R) for the pixel B(x1, y1) is less than that for the pixel A(x1, y1) and is close to 0. When the average μ of the discrepancy (R\hat{ }−R) for each pixel is calculated, the training function 124 calculates the frequency distribution p(μ_{R\hat{ }-R}) form the average μ of the pixels and calculates the distance (for example, the KL divergence distance D_{KL}) between the frequency distribution p(p_{R\hat{ }-R}) and a predetermined probability density distribution (for example, the normal distribution N(0, 1)). As expressed by Expression (1), a less loss is calculated as the discrepancy (R\hat{ }−R) in the first term becomes less and the distance D_{KL} between the distributions becomes less.

The loss function may be expressed by Expression (2) instead of Expression (1).

F=∥\hat{R}−R∥²+D_{KL}[p(\hat{R}(λ)−R(λ))∥N(0,1)] (2)

The first term in the first half stage of the loss function F in Expression (2) represents the magnitude of the discrepancy (R\hat{ }−R) between the spectral reflectance R\hat{ } and the spectral reflectance R as in Expression (1) and may be expressed, for example, by a mean square error or a mean absolute error. The second term in the second half stage of the loss function F in Expression (2) represents a distance D_{KL} between a frequency distribution p(R\hat{ }(λ)−R(λ)) and a normal distribution N(0, 1) when a discrepancy (R\hat{ }(λ)−R(λ)) for each wavelength λ at the time of visualization of the edema image is calculated and it is assumed that the frequency distribution p (R\hat{ }(λ)−R(λ)) of the discrepancy (R\hat{ }(λ)−R(λ)) conforms to the normal distribution N(0, 1) with an average 0 and a variance 1. The distance D_{KL} between the distributions indicates a KL divergence distance.

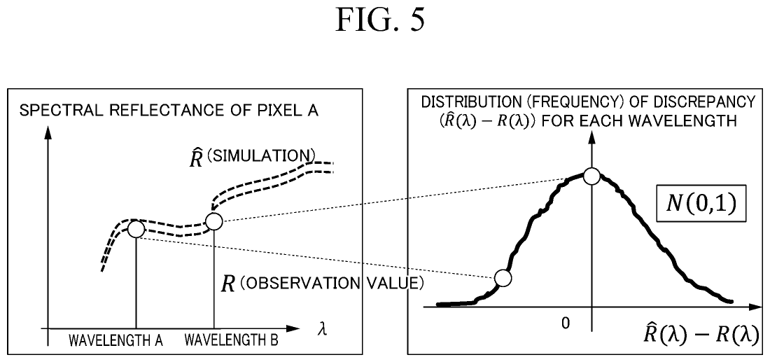

FIG. 5 is a diagram illustrating a frequency distribution p(R\hat{ }(λ)−R(λ)) of the discrepancy (R\hat{ }(λ)−R(λ)) for each wavelength λ. For example, the training function 124 calculates the discrepancy (R\hat{ }(λ_{A})−R(λ_{A})) at a certain wavelength λ_{A} and the discrepancy (R\hat{ }(λ_{B})−R(λ_{B})) at a wavelength λ_{B} for the pixel A(x1, y1). In the example illustrated in the drawing, it can be seen that the discrepancy (R\hat{ }(λ)−R(λ)) at the wavelength λ_{B} is less than that at the wavelength λ_{A} and is close to almost 0. When the discrepancy (R\hat{ }(λ)−R(λ)) at each wavelength λ is calculated, the training function 124 calculates the frequency distribution p(R\hat{ }(λ)−R(λ)) from the discrepancies (R\hat{ }(λ)−R(λ)) at the wavelengths k and calculates the distance (for example, the KL divergence distance D_{KL}) between the frequency distribution p (R\hat{ }(λ)−R(λ)) and a predetermined probability density distribution (for example, the normal distribution N(0, 1)). As expressed by Expression (2), a less loss is calculated as the discrepancy (R\hat{ }−R) in the first term becomes less and the distance D_{KL} between the distributions becomes less.

The average of the normal distribution is not limited to 0, and the variance thereof is not limited to 1. The average or the variance may have an arbitrary value. The probability density distribution is not limited to the normal distribution and may be another probability density distribution such as a mixed normal distribution. The number of probability density distributions is not limited to one, a plurality of probability density distributions may be prepared and learned using parameters, and parameters most applicable to a model may be employed. The distance between distributions is not limited to the KL divergence distance D_{KL} and may be a distance based on a histogram cross-over method or an arbitrary other distance.

Description with reference to the flowchart will be continued. Then, when a loss is calculated, the training function 124 trains the machine learning model MDL1 on the basis of the loss (Step S110).

For example, when the machine learning model MDL1 is implemented using a neural network, the training function 124 adjusts parameters (weighting factors or bias components) of the neural network such that the loss decreases using a stochastic gradient descent method or a steepest descent method. When the machine learning model MDL1 is implemented using a genetic algorithm, the training function 124 adjusts parameters (such as a crossover rate, a mutation rate, a selection pattern, and a crossover pattern) of the genetic algorithm such that the loss decreases.

Then, the training function 124 determines whether the loss converges on a fixed value (Step S112), and returns the routine to Step S102 when the loss does not converge on the fixed value. Accordingly, the first estimation function 122 selects a pixel other than the previously selected pixel out of a plurality of pixels included in the edema image again and estimates an in-vivo component volume (C_{Hb}, CH₂O, and Cₘₑₗ) from the spectral reflectance R corresponding to the pixel value of the selected pixel. In this way, selection of a pixel and training of the machine learning model MDL1 are repeated until the loss converges on a fixed value.

On the other hand, when the loss converges on a fixed value, the training function 124 updates the first model information in the memory 114 with definition of the machine learning model MDL1 when the loss has converged on a fixed value (that is, the trained machine learning model MDL1) and ends the routine of the flowchart (a series of processes for training the machine learning model MDL1).

[Process Flow and Runtime of Information Processing Device]

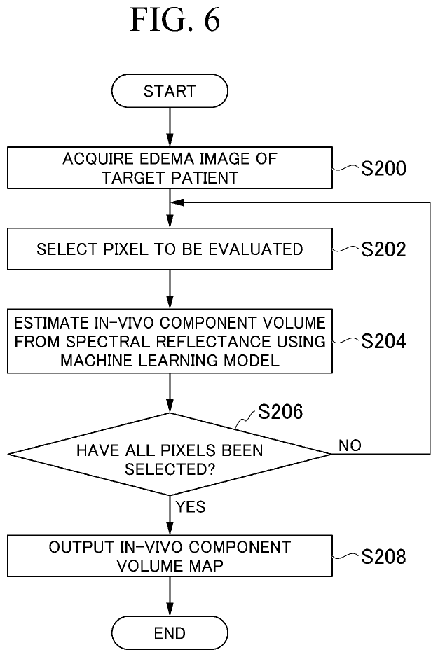

FIG. 6 is a flowchart illustrating a flow of a series of processes which are performed by the processing circuitry 120 according to the embodiment. The routine of the flowchart illustrated in FIG. 6 is performed when training the machine learning model MDL1 has been completed.

First, the acquisition function 121 acquires an edema image of a patient to be diagnosed (a patient with an edema) (Step S200). As described above, when the edema image is acquired, the acquisition function 121 may perform image processing such as a smoothing filter or edge extraction on the edema image.

Then, the first estimation function 122 selects one pixel to be evaluated out of a plurality of pixels included in the edema image acquired by the acquisition function 121 (Step S202).

Then, the first estimation function 122 estimates an in-vivo component volume (C_{Hb}, CH₂O, and Cₘₑₗ) from a spectral reflectance R which is a pixel value of the pixel to be evaluated using the machine learning model MDL1 defined by the first model information in the memory 114 (the trained machine learning model MDL1) (Step S204). In other words, the first estimation function 122 converts (encodes) the spectral reflectance R which is a pixel value of the pixel to be evaluated to the in-vivo component volume (C_{Hb}, CH₂O, and Cₘₑₗ) using the trained machine learning model MDL1.

Then, the first estimation function 122 determines whether all pixels included in the edema image have been selected as an evaluation target (Step S206).

When all pixels have not been selected as an evaluation target, the first estimation function 122 returns the routine to Step S202. That is, the first estimation function 122 selects a pixel other than the previously selected pixel again and estimates the in-vivo component volume (C_{Hb}, CH₂O, and Cₘₑₗ) from the spectral reflectance R which is a pixel value of the selected pixel. In this way, the routine is repeated until the in-vivo component volume (C_{Hb}, CH₂O, and Cₘₑₗ) is estimated from the spectral reflectance R of all the pixels.

On the other hand, when all the pixels have been selected as an evaluation target, the output control function 125 generates an image (hereinafter referred to as an in-vivo component volume map) in which the pixel values of all the pixels are replaced with the in-vivo component volumes (C_{Hb}, CH₂O, and Cₘₑₗ) and outputs the generated in-vivo component volume map via the output interface 113 (Step S208).

For example, the output control function 125 may display the in-vivo component volume map on the display 113a. The output control function 125 may transmit the in-vivo component volume map to an external device (for example, a computer used by a doctor in charge of the patient to be diagnosed) via the communication interface 111. In this way, the routine of the flowchart ends.

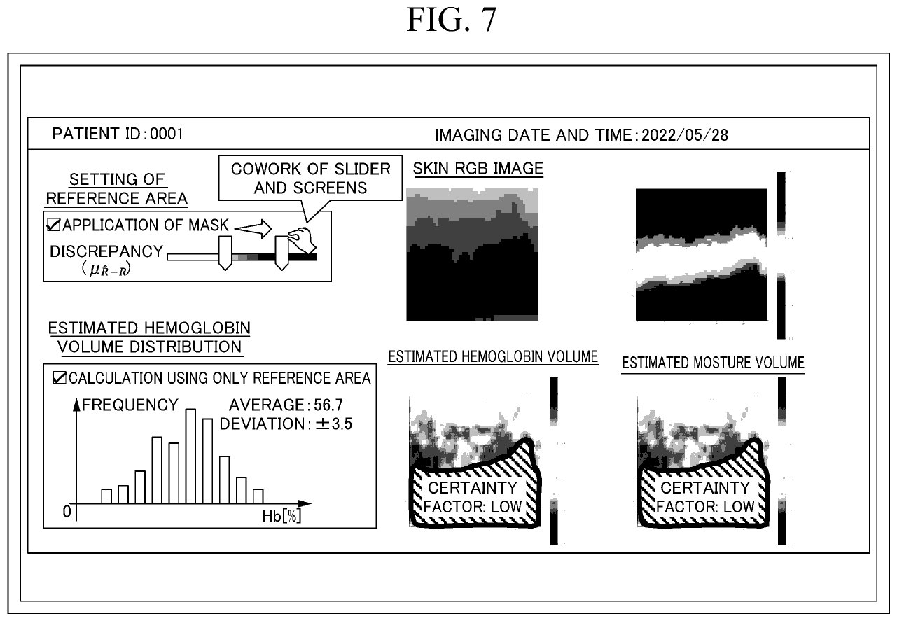

FIGS. 7 to 9 are diagrams illustrating an example of a screen of the display 113a on which the in-vivo component volume map is displayed. For example, the discrepancy between the spectral reflectance R observed using the camera or the like and the spectral reflectance R\hat{ } simulated using the numerical simulation model MDL2 or the in-vivo component volume (C_{Hb}, CH₂O, and Cₘₑₗ) before it has been decoded to the spectral reflectance R\hat{ } may be displayed on the screen of the display 113a. For example, the output control function 125 may display a message indicating that a certainty factor of the in-vivo component volume (C_{Hb}, CH₂O, and Cₘₑₗ) estimated using the machine learning model MDL1 is higher as the discrepancy becomes less and the certainty factor of the in-vivo component volume (C_{Hb}, CH₂O, and Cₘₑₗ) estimated using the machine learning model MDL1 is lower as the discrepancy becomes greater, on the screen of the display 113a. Accordingly, it is possible to enable more accurate diagnosis.

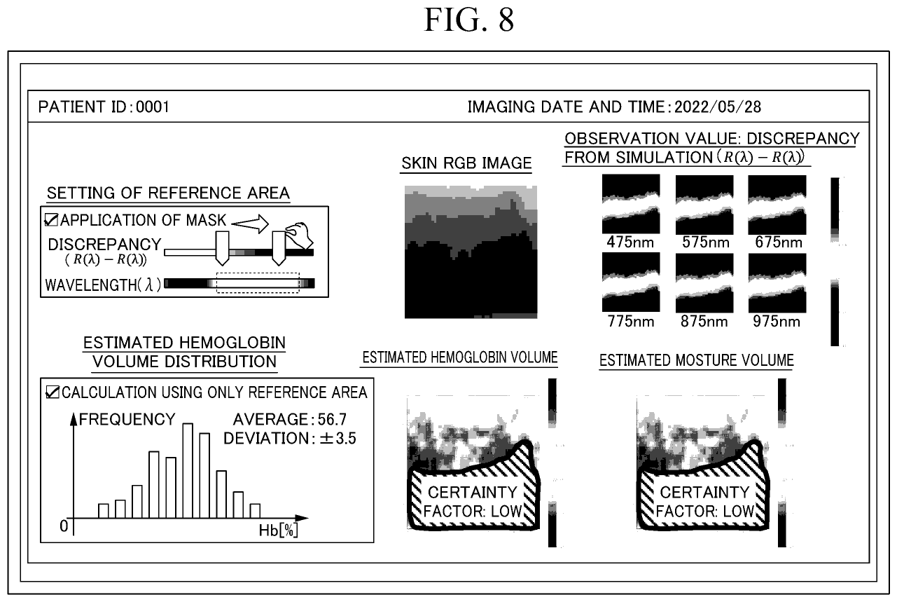

For example, the output control function 125 may display the average μ_{R\hat{ }-R} of the discrepancy for each pixel as the certainty factor on the display 113a as illustrated in FIG. 7. The output control function 125 may display the (R\hat{ }(λ)−R(λ)) for each wavelength λ as the certainty factor on the display 113a as illustrated in FIG. 8. The output control function 125 may display the discrepancy in a certain area (a set of some pixels) as the certainty factor on the display 113a.

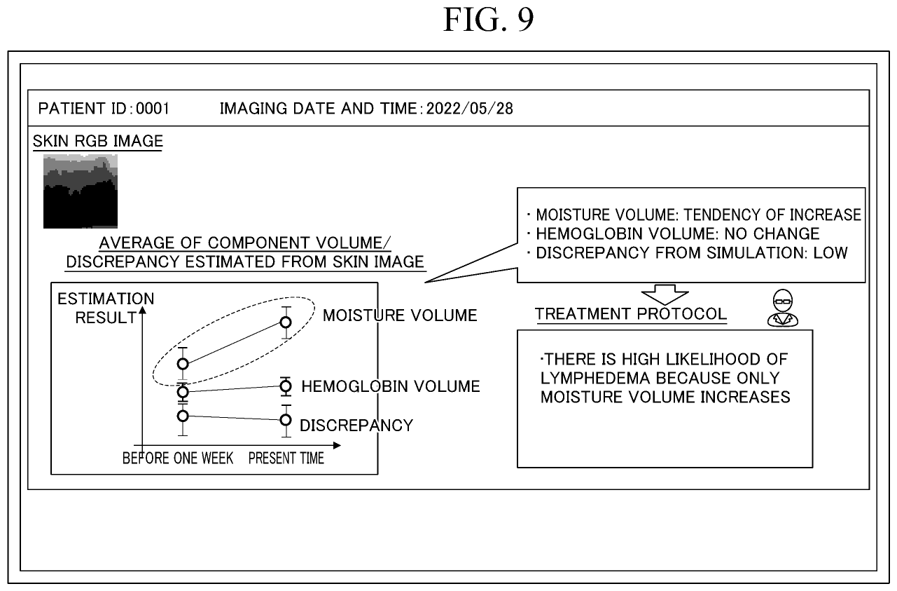

As illustrated in FIG. 9, the output control function 125 may compare the past in-vivo component volumes (C_{Hb}, CH₂O, and Cₘₑₗ) and the past discrepancy (certainty factor) with the current in-vivo component volume (C_{Hb}, CH₂O, and Cₘₑₗ) and the current discrepancy (certainty factor) and display the comparison result on the display 113a.

According to the aforementioned embodiment, the processing circuitry 120 of the information processing device 100 converts (encodes) a spectral reflectance R of each pixel in an edema image to an in-vivo component volume (C_{Hb}, CH₂O, and Cₘₑₗ) using the machine learning model MDL1. The processing circuitry 120 restores (decodes) the spectral reflectance R\hat{ } from the in-vivo component volume (C_{Hb}, CH₂O, and Cₘₑₗ) using the numerical simulation model MDL2. Then, the processing circuitry 120 trains the machine learning model MDL1 on the basis of the discrepancy between the spectral reflectance R has not been converted to the in-vivo component volume (C_{Hb}, CH₂O, and Cₘₑₗ) and the spectral reflectance R\hat{ } restored from the in-vivo component volume (C_{Hb}, CH₂O, and Cₘₑₗ). By estimating the in-vivo component volume (C_{Hb}, CH₂O, and Cₘₑₗ) using the trained machine learning model MDL1, it is possible to provide information with a higher certainty factor even when there may be a discrepancy between an actual phenomenon and a simulation result. As a result, a doctor or a patient can accurately recognize a target event such as an edema.

### Other Embodiments

Other embodiments will be described below. As described above, the information processing device 100 according to the aforementioned embodiment can be applied to other events in addition to an event such as an “edema.” For example, in a radiographic treatment planning program (a radiographic treatment device), the information processing device 100 according to the aforementioned embodiment can be applied to a situation in which simulation (for example, a Monte Carlo method) is performed to identify geometric parameters (an irradiation angle, a position) in which a target region of a patient to be treated can be irradiated.

In the aforementioned embodiment, the spectral reflectance R\hat{ } is restored (decoded) from the in-vivo component volume (C_{Hb}, CH₂O, and Cₘₑₗ) using the numerical simulation model MDL2 which is implemented on the basis of a Monte Carlo method, the Kubelka-Munk theory, or the Lambert-Beer law, but the present invention is not limited thereto. For example, the spectral reflectance R\hat{ } may be restored from the in-vivo component volume (C_{Hb}, CH₂O, and Cₘₑₗ) using a second machine learning model. The second machine learning model may be implemented using a neural network or a genetic algorithm similarly to the machine learning model MDL1. That is, an encoder and a decoder may be implemented using a neural network or a genetic algorithm.

While some embodiments have been described above, the embodiments are presented only as examples and are not intended to limit the scope of the invention. The embodiments can be modified in various other forms, and various omissions, substitutions, and alterations can be performed thereon without departing from the gist of the invention. These embodiments or modifications thereof are included in the scope or gist of the invention and are included in the inventions described in the appended claims and scopes equivalent thereto.

What is claimed is:

1. An information processing device comprising processing circuitry configured to:
acquire observation data which is acquired when a target event is observed;
convert the observation data to a feature quantity of the target event using a machine learning model;
restore the observation data from the feature quantity using a numerical simulation model; and
train the machine learning model on the basis of a discrepancy between first observation data which is the observation data that has not been converted to the feature quantity and second observation data which is the observation data restored from the feature quantity.

2. The information processing device according to claim 1, wherein the processing circuitry trains the machine learning model on the basis of a loss function based on the assumption that a frequency distribution of the discrepancy conforms to a predetermined probability density distribution.

3. The information processing device according to claim 2, wherein the observation data is a spectral reflectance which is acquired from an image obtained by imaging a patient's skin,
wherein the feature quantity is an in-vivo component volume of the patient, and
wherein the loss function is a function for calculating the discrepancy for each pixel included in the image, the function being based on the assumption that the frequency distribution of the discrepancy for each pixel conforms to the predetermined probability density distribution.

4. The information processing device according to claim 2, wherein the observation data is a spectral reflectance which is acquired from an image obtained by imaging a patient's skin,
wherein the feature quantity is an in-vivo component volume of the patient, and
wherein the loss function is a function for calculating the discrepancy for each wavelength when the image is visualized, the function being based on the assumption that the frequency distribution of the discrepancy for each wavelength conforms to the predetermined probability density distribution.

5. The information processing device according to claim 2, wherein the loss function is a function for outputting a smaller loss as the discrepancy becomes less and as a distance between the frequency distribution of the discrepancy and the predetermined probability density distribution becomes less, and
wherein the processing circuitry trains the machine learning model such that the loss decreases.

6. The information processing device according to claim 1, wherein the machine learning model is a model using a neural network or a genetic algorithm, and
wherein the numerical simulation model is a model using a Monte Carlo method, the Kubelka-Munk theory, or the Lambert-Beer law.

7. An information processing method which is performed by a computer, the information processing method comprising:
acquiring observation data which is acquired when a target event is observed;
converting the observation data to a feature quantity of the target event using a machine learning model;
restoring the observation data from the feature quantity using a numerical simulation model; and
training the machine learning model on the basis of a discrepancy between first observation data which is the observation data that has not been converted to the feature quantity and second observation data which is the observation data restored from the feature quantity.

8. A non-transitory computer-readable storage medium storing a program, the program causing a computer to perform:
acquiring observation data which is acquired when a target event is observed;
converting the observation data to a feature quantity of the target event using a machine learning model;
restoring the observation data from the feature quantity using a numerical simulation model; and
training the machine learning model on the basis of a discrepancy between first observation data which is the observation data that has not been converted to the feature quantity and second observation data which is the observation data restored from the feature quantity.
